(19) United States
(12) Patent Application Publication
(10) Pub. No.: US 2012/0264634 A1
(43) Pub. Date: Oct. 18, 2012

(54) Marker Sequences for Pancreatic Cancer Diseases, Pancreatic Carcinoma and Use Thereof

(75) Inventors: Peter Amersdorfer, Graz (AT); Annabel Höpfner, Dortmund (DE); Angelika Lueking, Bochum (DE)

(73) Assignee: PROTAGEN Aktiengesellschaft, Dortmund (DE)

(21) Appl. No.: 13/498,964

(22) PCT Filed: Sep. 29, 2010
(86) PCT No.: PCT/EP2010/064510

§ 371 Date: Jun. 22, 2012

(30) Foreign Application Priority Data
Sep. 29, 2009 (EP) 09171690.2

(51) Int. Cl. C40B 30/04; G01N 21/64; G01N 27/72; C07K 14/435; C40B 40/06; C40B 40/10; C12N 5/09; C07H 21/04; G01N 33/574; G01N 27/62

(52) U.S. Cl. 506/9; 436/501; 435/6.14; 435/7.92; 506/16; 506/18; 435/2; 536/23.1; 530/350

(57) ABSTRACT

The present invention relates to novel marker sequences for pancreatic cancer diseases, pancreatic carcinoma and the diagnostic use thereof together with a method for screening of potential active substances for pancreatic cancer diseases, pancreatic carcinoma by means of these marker sequences. Furthermore, the invention relates to a diagnostic device containing such marker sequences for pancreatic cancer diseases, pancreatic carcinoma, in particular a protein biochip and the use thereof.

The present invention relates to novel marker sequences for pancreatic cancer diseases, pancreatic carcinoma, and the diagnostic use thereof together with a method for screening potential active substances for pancreatic cancer diseases of this type by means of these marker sequences. Furthermore, the invention relates to a diagnostic device containing marker sequences of this type for pancreatic cancer diseases, pancreatic carcinoma, in particular a protein biochip and the use thereof

The 5-year-survival rate for pancreatic carcinoma of approx. 1% is the lowest of all cancer types (Parkin, D. M., F. Bray, et al. (2001). “Estimating the world cancer burden: Globocan 2000.” Int J Cancer 94(2): 153-6). Early diagnosis might increase the 5-year survival rate to 40% (Yeo, C. J. and J. L. Cameron (1998). “Prognostic factors in ductal pancreatic cancer.” Langenbecks Arch Surg 383(2): 129-33). Therefore, for diagnosis, the precursor diseases of pancreatic cancer need to be considered as well, such as PDAC (pancreatic ductal adenocarcinoma), PanIN (pancreatic intraepithelial neoplasias), pancreatic lesions, CP (chronic pancreatitis), including endocrine tumors of the pancreas. Especially PanID are associated with pancreatic lesions and differentiate them morphologically into PanIn 1A, 1B, 2, and 3 (Kern, S., R. Hruban, et al. (2001). “A white paper: the product of a pancreas cancer think tank.” Cancer Res 61(12): 4923-32). Pancreatic lesions have also been described for CP. Endocrine (benign or malignant) tumors of the pancreas, particularly neuroendocrine tumors, are relevant as well.

WO2008064670 describes e.g. marker genes for pancreas obtained by means of proteome analysis and histological investigations.

Protein biochips are gaining increasing industrial importance in analysis and diagnosis as well as in pharmaceutical development. Protein biochips have become established as screening instruments.

The rapid and highly parallel detection of a multiplicity of specifically binding analysis molecules in a single experiment is rendered possible hereby. To produce protein biochips, it is necessary to have the required proteins available. For this purpose, in particular protein expression libraries have become established. The high throughput cloning of defined open reading frames is one possibility (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P., (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Reboul, J., Vaglio, P., Rual, J. F., Lamesch, P., Martinez, M., Armstrong, C M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome Version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A. , van den Heuvel, S. and Vidal, M. (2000) GATEWAY recombinational cloning: application to the cloning of large numbers of open reading frames or ORFeomes. Methods Enzymol, 328, 575-592). However, an approach of this type is strongly connected to the progress of the genome sequencing projects and the annotation of these gene sequences. Furthermore, the determination of the expressed sequence can be ambiguous due to differential splicing processes. This problem may be circumvented by the application of cDNA expression libraries (Büssow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, K., Nordhoff, E., Lübbert, C, Lehrach, H. and Walter, G. (2000) A human cDNA library for high-throughput protein expression screening. Genomics, 65, 1-8; Holz, C, Lueking, A., Bovekamp, L., Gutjahr, C, Bolotina, N., Lehrach, H. and Cahill, D. J. (2001) A human cDNA expression library in yeast enriched for open reading frames. Genome Res, 11, 1730-1735; Lueking, A., Holz, C, Gotthold, C, Lehrach, H. and Cahill, D. (2000) A system for dual protein expression in Pichia pastoris and Escherichia coli, Protein Expr. Purif., 20, 372-378). The cDNA of a particular tissue is hereby cloned into a bacterial or an eukaryotic expression vector, such as, e.g., yeast. The vectors used for the expression are generally characterized in that they carry inducible promoters that may be used to control the time of protein expression. Furthermore, expression vectors have sequences for so-called affinity epitopes or affinity proteins, which on the one hand permit the specific detection of the recombinant fusion proteins by means of an antibody directed against the affinity epitope, and on the other hand the specific purification via affinity chromatography (IMAC) is rendered possible.

For example, the gene products of a cDNA expression library from human fetal brain tissue in the bacterial expression system Escherichia coli were arranged in high-density format on a membrane and could be successfully screened with different antibodies. It was possible to show that the proportion of full-length proteins is at least 66%. Additionally, the recombinant proteins from the library could be expressed and purified in a high-throughput manner (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Büssow (2000) supra; Lueking, A., Horn, M., Eickhoff, H., Büssow, K., Lehrach, H. and Walter, G. (1999) Protein microarrays for gene expression and antibody screening. Analytical Biochemistry, 270, 103-111). Protein biochips of this type based on cDNA expression libraries are in particular the subject matter of WO 99/57311 and WO 99/57312.

Furthermore, in addition to antigen-presenting protein biochips, antibody-presenting arrangements are likewise described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

Protein-biochips have a advantageously high sensitivity.

However, there is a great need to provide indication-specific diagnostic devices, such as a protein biochip.

The object of the present invention is therefore to provide improved marker sequences and the diagnostic use thereof for the treatment of pancreatic cancer diseases up to pancreatic carcinoma.

The object of the present invention is solved in providing of novel marker sequences SEQ 1-1004, which are identified for the first time by means of a protein biochip, in particular including bioinformatic analysis. Therefore, SEQ 1-1004 are identified for the first time by means of a protein biochip.

The provision of specific marker sequences permits a reliable diagnosis and stratification of patients with pancreatic cancer diseases up to pancreatic carcinoma, in particular by means of a protein biochip.

The invention therefore relates to the use of marker sequences for the diagnosis of pancreatic cancer diseases up to pancreatic carcinoma, wherein at least one marker sequence of a cDNA selected from the group SEQ 1-1004 or respectively a protein coding therefor or respectively a partial sequence or fragment thereof (hereinafter: marker sequences according to the invention) is determined on or from a patient to be examined

It was possible to identify the marker sequences according to the invention by means of differential screening of samples from healthy test subjects with patient samples with pancreatic cancer diseases, pancreatic carcinoma.

The term “pancreatic cancer diseases up to pancreatic carcinoma” in accordance with the invention encompasses also precursor and/or concomitant illnesses thereof, pancreatic cancer diseases and/or pancreatic carcinoma as such, however in particular PDAC (Pancreatic ductal adenocarcinoma), PanIN (pancreatic intraepithelial neoplasias), pancreatic lesions, CP (chronic pancreatitis), including endocrine pancreatic tumors, particularly pancreatic tumors and pancreatic neoplasm (definition, e.g., according to Pschyrembel, de Gruyter, 261st edition (2007), Berlin).

In a further embodiment at least 2 to 5 or 10, preferably 30 to 50 marker sequences, or 50 to 100 or more marker sequences are determined on or from a patient to be examined

In a further embodiment of the invention, the marker sequences according to the invention can likewise be combined, supplemented, fused, or expanded likewise with known biomarkers for this indication.

In a preferred embodiment, the determination of the marker sequences is carried out outside the human body and the determination is carried out in an ex vivo/in vitro diagnosis.

In a further embodiment of the invention, the invention relates to the use of marker sequences as diagnostic agents, wherein at least one marker sequence of a cDNA is selected from the group SEQ 1-1004, preferably SEQ 503-1004, or respectively a protein coding therefor or respectively a partial sequence or fragment thereof.

Furthermore, the invention relates to a method for the diagnosis of pancreatic cancer diseases up to pancreatic carcinoma, wherein a.) at least one marker sequence of a cDNA selected from the group SEQ 1-1004, preferably SEQ 503-1004, or respectively a protein coding therefor or respectively a partial sequence or fragment thereof is applied to a solid support and b.) is brought into contact with body fluid or tissue extract of a patient and c.) the detection of an interaction of the body fluid or tissue extract with the marker sequences from a.) is carried out.

The invention therefore likewise relates to diagnostic agents for the diagnosis of pancreatic cancer diseases up to pancreatic carcinoma respectively selected from the group SEQ 1-1004, preferably SEQ 503-1004, or respectively a protein coding therefor or respectively a partial sequence or fragment thereof

The detection of an interaction of this type can be carried out, for example, by a probe, in particular by an antibody.

The invention therefore likewise relates to the object of providing a diagnostic device or an assay, in particular a protein biochip, which permits a diagnosis or examination for pancreatic cancer diseases up to pancreatic carcinoma.

Furthermore, the invention relates to a method for the stratification, in particular risk stratification and/or therapy control of a patient with pancreatic cancer diseases up to pancreatic carcinoma, wherein at least one marker sequence of a cDNA selected from the group 1-1004, preferably SEQ 503-1004, or respectively a protein coding therefor is determined on a patient to be examined

Furthermore, the stratification of the patients with pancreatic cancer diseases up to pancreatic carcinoma in new or established subgroups of inflammatory pancreatic cancer diseases up to pancreatic carcinoma is also covered, as well as the expedient selection of patient groups for the clinical development of novel therapeutic agents. The term therapy control likewise covers the allocation of patients to responders and non-responders regarding a therapy or the therapy course thereof.

“Diagnosis” for the purposes of this invention means the positive determination of pancreatic cancer diseases up to pancreatic carcinoma by means of the marker sequences according to the invention as well as the assignment of the patients to pancreatic cancer diseases up to pancreatic carcinoma. The term diagnosis covers medical diagnostics and examinations in this regard, in particular in-vitro diagnostics and laboratory diagnostics, likewise proteomics and nucleic acid blotting. Further tests can be necessary to be sure and to exclude other diseases. The term diagnosis therefore likewise covers the differential diagnosis of pancreatic cancer diseases, pancreatic carcinoma by means of the marker sequences according to the invention and the prognosis of pancreatic cancer diseases, pancreatic carcinoma.

“Stratification or therapy control” for the purposes of this invention means that the method according to the invention renders possible decisions for the treatment and therapy of the patient, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof

In a further embodiment of the invention, the term “stratification” covers in particular the risk stratification with the prognosis of an outcome of a negative health event.

Within the scope of this invention, “patient” means any test subject—human or mammal—with the proviso that the test subject is tested for pancreatic cancer diseases up to pancreatic carcinoma.

The term “marker sequences” for the purposes of this invention means that the cDNA or the polypeptide or protein that can be respectively obtained therefrom are significant for pancreatic cancer diseases, pancreatic carcinoma. For example, the cDNA or the polypeptide or protein that can be respectively obtained therefrom can exhibit an interaction with substances from the body fluid or tissue extract of a patient with pancreatic cancer diseases, pancreatic carcinoma (e.g., antigen (epitope)/antibody (paratope) interaction). For the purposes of the invention “wherein at least one marker sequence of a cDNA selected from the group SEQ 1-1004, preferably SEQ 503-1004 or respectively a protein coding therefor or respectively a partial sequence or fragment thereof is determined on a patient to be examined” means that an interaction between the body fluid or tissue extract of a patient and the marker sequences according to the invention is detected. An interaction of this type is, e.g., a bond, in particular a binding substance on at least one marker sequence according to the invention or in the case of a cDNA the hybridization with a suitable substance under selected conditions, in particular stringent conditions (e.g., such as usually defined in J. Sambrook, E. F. Fritsch, T. Maniatis (1989), Molecular cloning: A laboratory manual, 2nd Edition, Cold Spring Harbor Laboratory Press, Cold Spring Harbor, USA or Ausubel, “Current Protocols in Molecular Biology,” Green Publishing Associates and Wiley Interscience, N.Y. (1989)). One example of stringent hybridization conditions is: hybridization in 4×SSC at 65° C. (alternatively in 50% formamide and 4×SSC at 42° C.), followed by several washing steps in 0.1×SSC at 65° C. for a total of approximately one hour. An example of less stringent hybridization conditions is hybridization in 4×SSC at 37° C., followed by several washing steps in 1×SSC at room temperature.

According to the invention, substances of this type are constituents of a body fluid, in particular blood, whole blood, blood plasma, blood serum, patient serum, urine, cerebrospinal fluid, synovial fluid, or of a tissue extract of the patient.

In a further embodiment of the invention, however, the marker sequences according to the invention can be present in a significantly higher or lower expression rate or concentration that indicates pancreatic cancer diseases, pancreatic carcinoma. The relative sick/healthy expression rates of the marker sequences for pancreatic cancer diseases, pancreatic carcinoma according to the invention are hereby determined by means of a protein biochip according to the invention.

In a further embodiment of the invention, the marker sequences have a recognition signal that is addressed to the substance to be bound (e.g., antibody, nucleic acid). It is preferred according to the invention that for a protein the recognition signal is an epitope and/or a paratope and/or a hapten and for a cDNA is a hybridization or binding region.

The marker sequences according to the invention are the subject matter of Table A and can be clearly identified by the respectively cited database entry (also by means of the Internet: http://www.ncbi.nlm.nih.gov/) (see in Table A), see also the associated sequence protocol.

The sequences 1 to 502 relate to the full-length sequences of the sequences 503 to 1004. The sequences 503 to 1004 are preferred sequences, which are identified directly from the protein biochip according to the invention.

According to the invention, the marker sequences also cover those modifications of the cDNA sequence and the corresponding amino acid sequence as chemical modification, such as citrullination, acetylation, phosphorylation, glycosylation or poly(A) strand and other modifications known to one skilled in the art.

In a further embodiment of the invention, partial sequences or fragments of the marker sequences according to the invention are likewise comprised. In particular those partial sequences that have an identity of 95%, 90%, in particular 80% or 70% with the marker sequences according to the invention.

Such partial sequences or fragments of the marker sequences according to the invention are defined by function and have the same diagnostic function.

Partial sequences are also sequences of the type which have 50 to 100 nucleotides, 70-120 nucleotides of a sequence of the SEQ 1-1004, preferably SEQ 503-1004, or peptides obtainable therefrom.

In a further embodiment, the respective marker sequence can be represented in different quantities in one more regions on a solid support. This permits a variation of the sensitivity.

The regions can have respectively a totality of marker sequences, i.e., a sufficient number of different marker sequences, in particular 2 to 5 or 10 or more and optionally more nucleic acids and/or proteins, in particular biomarkers. However, at least 96 to 25,000 (numerical) or more from different or identical marker sequences and further nucleic acids and/or proteins, in particular biomarkers are preferred. Furthermore preferred are more than 2,500, in particular preferred 10,000 or more different or identical marker sequences and optionally further nucleic acids and/or proteins, in particular biomarkers.

Another object of the invention relates to an arrangement of marker sequences containing at least one marker sequence of a cDNA selected from the group SEQ 1-1004, preferably SEQ 503-1004 or respectively a protein coding therefor. Preferably, the arrangement contains at least 2 to 5 or 10, preferably 30 to 50 marker sequences, or 50 to 100 or more marker sequences.

Within the scope of this invention, “arrangement” is synonymous with “array,” and if this “array” is used to identify substances on marker sequences, this is to be understood to be an “assay” or diagnostic device. In a preferred embodiment, the arrangement is designed such that the marker sequences represented on the arrangement are present in the form of a grid on a solid support. Furthermore, those arrangements are preferred that permit a high-density arrangement of protein binders and the marker sequences are spotted. Such high-density spotted arrangements are disclosed, for example, in WO 99/57311 and WO 99/57312 and can be used advantageously in a robot-supported automated high-throughput method.

Within the scope of this invention, however, the term “assay” or diagnostic device likewise comprises those embodiments of a device, such as ELISA, bead-based assay, line assay, Western Blot, immunochromatographic methods (e.g., so-called lateral flow immunoassays, or similar immunological single or multiplex detection measures. A protein biochip in terms of this invention is the systematic arrangement of proteins on a solid support.

The marker sequences of the arrangement are fixed on a solid support, but preferably spotted or immobilized even printed on, i.e. applied in a reproducible manner. One or more marker sequences can be present multiple times in the totality of all marker sequences and present in different quantities based on one spot. Furthermore, the marker sequences can be standardized on the solid support (i.e., by means of serial dilution series of, e.g., human globulins as internal calibrators for data normalization and quantitative evaluation).

The invention therefore relates to an assay or a protein biochip comprising an arrangement containing marker sequences according to the invention.

In a further embodiment, the marker sequences are present as clones. Clones of this type can be obtained, for example, by means of a cDNA expression library according to the invention (Büssow et al. 1998 (supra)). In a preferred embodiment, such expression libraries containing clones are obtained using expression vectors from a cDNA expression library comprising the cDNA marker sequences. These expression vectors preferably contain inducible promoters. The induction of the expression can be carried out, e.g., by means of an inductor, such as IPTG. Suitable expression vectors are described in Terpe et al. (Terpe T Appl Microbiol Biotechnol. 2003 January; 60(5): 523-33).

One skilled in the art is familiar with expression libraries, they can be produced according to standard works, such as Sambrook et al, “Molecular Cloning, A laboratory handbook, 2nd edition (1989), CSH press, Cold Spring Harbor, N.Y. Expression libraries are also preferred which are tissue-specific (e.g., human tissue, in particular human organs).

Furthermore included according to the invention are expression libraries that can be obtained by exon-trapping. A synonym for expression library is expression bank.

Also preferred are protein biochips or corresponding expression libraries that do not exhibit any redundancy (so-called: Uniclone® library) and that may be produced, for example, according to the teachings of WO 99/57311 and WO 99/57312. These preferred Uniclone libraries have a high portion of non-defective fully expressed proteins of a cDNA expression library.

Within the context of this invention, the clones can also be, but not limited to, transformed bacteria, recombinant phages, or transformed cells from mammals, insects, fungi, yeasts, or plants.

The clones are fixed, spotted, or immobilized on a solid support.

The invention therefore relates to an arrangement wherein the marker sequences are present as clones.

Additionally, the marker sequences can be present in the respective form of a fusion protein, which contains, for example, at least one affinity epitope or tag. The tag may be one such as contains c-myc, his tag, arg tag, FLAG, alkaline phosphatase, VS tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase, or lacZ.

In all of the embodiments, the term “solid support” covers embodiments such as a filter, a membrane, a magnetic or fluorophore-labeled bead, a silica wafer, glass, metal, ceramics, plastics, a chip, a target for mass spectrometry, or a matrix. However, a filter is preferred according to the invention.

As a filter, furthermore PVDF, nitrocellulose, or nylon is preferred (e.g, Immobilon P Millipore, Protran Whatman, Hybond N+ Amersham).

In another preferred embodiment of the arrangement according to the invention, the arrangement corresponds to a grid with the dimensions of a microtiter plate (8-12 wells strips, 96 wells, 384 wells, or more), a silica wafer, a chip, a target for mass spectrometry, or a matrix.

In a further embodiment, the invention relates to an assay or a protein biochip for identifying and characterizing a substance for pancreatic cancer diseases, pancreatic carcinoma, characterized in that an arrangement or assay according to the invention is a.) brought into contact with at least one substance to be tested and b.) a binding success is detected.

Furthermore, the invention relates to a method for identifying and characterizing a substance for pancreatic cancer diseases, pancreatic carcinoma, characterized in that an arrangement or assay according to the invention is a.) brought into contact with at least one substance to be tested and b.) a binding success is detected.

The substance to be tested can be any native or non-native biomolecule, a synthetic chemical molecule, a mixture, or a substance library.

After the substance to be tested contacts a marker sequence, the binding success is evaluated, which, for example, is carried out using commercially available image analyzing software (GenePix Pro (Axon Laboratories), Aida (Ray test), ScanArray (Packard Bioscience)).

The visualization of protein-protein interactions according to the invention (e.g., protein on marker sequence, as antigen/antibody) or corresponding “means for detecting the binding success” can be performed, for example, using fluorescence labeling, biotinylation, radioisotope labeling, or colloid gold or latex particle labeling in the usual way. A detection of bound antibodies is carried out with the aid of secondary antibodies, which are labeled with commercially available reporter molecules (e.g., Cy, Alexa, Dyomics, FITC, or similar fluorescent dyes, colloidal gold or latex particles), or with reporter enzymes, such as alkaline phosphatase, horseradish peroxidase, etc., and the corresponding colorimetric, fluorescent, or chemiluminescent substrates. Readout is conducted, e.g., using a microarray laser scanner, a CCD camera, or visually.

In a further embodiment, the invention relates to a drug/active substance or prodrug developed for pancreatic cancer diseases, pancreatic carcinoma and obtainable through the use of the assay or protein biochip according to the invention.

The invention therefore likewise relates to the use of an arrangement according to the invention or an assay for screening active substances for pancreatic cancer diseases, pancreatic carcinoma.

In a further embodiment, the invention therefore likewise relates to a target for the treatment and therapy of pancreatic cancer diseases, pancreatic carcinoma respectively selected from the group SEQ 1-1004, preferably SEQ 503-1004, or a protein respectively coding therefor.

In a further embodiment, the invention likewise relates to the use of the marker sequences according to the invention, preferably in the form of an arrangement, as an affinity material for carrying out an apheresis or in the broadest sense a blood lavage, wherein substances from body fluids of a patient with pancreatic cancer diseases, pancreatic carcinoma, such as blood or plasma, bind to the marker sequences according to the invention and consequently can be selectively withdrawn from the body fluid.

### EXAMPLES AND FIGURES

Ten or more patient samples were individually screened against a cDNA expression library. The expression clones specific to pancreatic cancer diseases, pancreatic carcinoma were determined through a comparison with ten or more healthy samples. The identity of the marker sequences was determined by DNA sequencing.

FIG. 1 shows the differential screening between two protein biochips from respectively one cDNA expression bank of a patient and a healthy test subject. The differential clones are detected by means of fluorescent labeling and evaluated by means of bioinformatics.

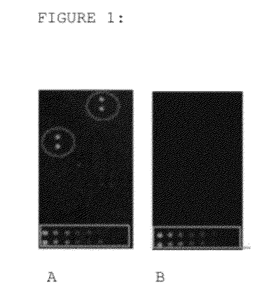

In the scope of the biomarker identification, various bioinformatic analyses are performed. For each serum, reactivities against approximately 2000 different antigens are measured by means of microarray. These data are used for a ranking of the spotted antigens with respect to their differentiation capability between healthy and diseased sera. This analysis is performed by means of the non-parameterized Mann-Whitney test on normalized intensity data. An internal standard which is also spotted on each chip is used for the normalization. Since a p value is calculated for each antigen, methods are used for correction of the multiple test. As a very conservative approach, a Bonferroni direction is performed and the less restrictive false discovery rate (FDR) according to Benjamini & Hochberg is additionally calculated. Furthermore, the data are used for classification of the sera. Different multivariate methods are used hereby. These are methods from statistical learning methods such as support vector machines (SVM), neural networks, or classification trees, as well as a threshold value method, which is capable of both classification and also visual representation of the data.

To avoid overfitting, a 10-fold cross-validation of the data is performed.

TABLE A

| SEQ | Accession No | Accession No | Name |
|---|---|---|---|
| 1 | gi\|10518498 | 11237 | Homo sapiens ring finger protein 24 (RNF24), mRNA |
| 2 | gi\|11038650 | 3550 | Homo sapiens IK cytokine, down-regulator of HLA II (IK), mRNA |
| 3 | gi\|221139925 | 57224 | Homo sapiens NHS-like 1, transcript variant 5 (NHSL1), mRNA |
| 4 | gi\|113428396 | 25759 | Homo sapiens SHC (Src homology 2 domain containing) transforming protein 2 (SHC2), mRNA |
| 5 | gi\|113430465 | 392485 | PREDICTED: Homo sapiens similar to ataxin 7-like 3 (LOC392485), mRNA |
| 6 | gi\|14249519 | 84908 | Homo sapiens hypothetical protein FLJ14668 (FLJ14668), mRNA |
| 7 | gi\|19913395 | 6525 | Homo sapiens smoothelin (SMTN), transcript variant 2, mRNA |
| 8 | gi\|21361156 | 9454 | Homo sapiens homer homolog 3 (Drosophila) (HOMER3), mRNA |
| 9 | gi\|21735620 | 4191 | Homo sapiens malate dehydrogenase 2, NAD (mitochondrial) (MDH2), mRNA |
| 10 | gi\|22027484 | 51655 | Homo sapiens RAS, dexamethasone-induced 1 (RASD1), mRNA |
| 11 | gi\|22027497 | 23523 | Homo sapiens calcineurin binding protein 1 (CABIN1), mRNA |
| 12 | gi\|22095372 | 80774 | Homo sapiens LIM domain containing 2 (LIMD2), mRNA |
| 13 | gi\|22538469 | 8320 | Homo sapiens eomesodermin homolog (Xenopus laevis) (EOMES), mRNA |
| 14 | gi\|22748870 | 136051 | Homo sapiens zinc finger protein 786 (ZNF786), mRNA |
| 15 | gi\|28178831 | 3418 | Homo sapiens isocitrate dehydrogenase 2 (NADP+), mitochondrial (IDH2), mRNA |
| 16 | gi\|29826322 | 118 | Homo sapiens adducin 1 (alpha) (ADD1), transcript variant 3, mRNA |
| 17 | gi\|31377576 | 143282 | Homo sapiens chromosome 10 open reading frame 13 (C10orf13), mRNA |
| 18 | gi\|32401423 | 65108 | Homo sapiens MARCKS-like 1 (MARCKSL1), mRNA |
| 19 | gi\|34147459 | 92922 | Homo sapiens coiled-coil domain containing 102A (CCDC102A), mRNA |
| 20 | gi\|34147626 | 65982 | Homo sapiens zinc finger protein 447 (ZNF447), mRNA |
| 21 | gi\|34147687 | 64777 | Homo sapiens required for meiotic nuclear division 5 homolog B (S. cerevisiae) (RMND5B), |
| | | | mRNA |
| 22 | gi\|34147700 | 147015 | Homo sapiens dehydrogenase/reductase (SDR family) member 13 (DHRS13), mRNA |
| 23 | gi\|34335280 | 5715 | Homo sapiens proteasome (prosome, macropain) 26S subunit, non-ATPase, 9 (PSMD9), mRNA |
| 24 | gi\|34452731 | 57580 | Homo sapiens phosphatidylinositol 3,4,5-trisphosphate-dependent RAC exchanger 1 (PREX1), |
| | | | mRNA |
| 25 | gi\|35250828 | 22820 | Homo sapiens coatomer protein complex, subunit gamma (COPG), mRNA |
| 26 | gi\|37550270 | | Homo sapiens chromosome 5 genomic contig, reference assembly |
| 27 | gi\|37551026 | | Homo sapiens chromosome 10 genomic contig, reference assembly |
| 28 | gi\|38679885 | 90864 | Homo sapiens splA/ryanodine receptor domain and SOCS box containing 3 (SPSB3), mRNA |
| 29 | gi\|38679886 | 6717 | Homo sapiens sorcin (SRI), transcript variant 1, mRNA |
| 30 | gi\|40353201 | 55593 | Homo sapiens OTU domain containing 5 (OTUD5), mRNA |
| 31 | gi\|40789263 | 84310 | Homo sapiens hypothetical protein MGC11257 (MGC11257), mRNA |
| 32 | gi\|40795668 | 10991 | Homo sapiens solute carrier family 38, member 3 (SLC38A3), mRNA |
| 33 | gi\|40804467 | 782 | Homo sapiens calcium channel, voltage-dependent, beta 1 subunit (CACNB1), transcript variant |
| | | | 1, mRNA |
| 34 | gi\|40807365 | 64118 | Homo sapiens dihydrouridine synthase 1-like (S. cerevisiae) (DUS1L), mRNA |
| 35 | gi\|41352714 | 55737 | Homo sapiens vacuolar protein sorting 35 (yeast) (VPS35), mRNA |
| 36 | gi\|42475533 | 9746 | Homo sapiens calsyntenin 3 (CLSTN3), mRNA |
| 37 | gi\|42476152 | 5764 | Homo sapiens pleiotrophin (heparin binding growth factor 8, neurite growth-promoting factor 1) |
| | | | (PTN), mRNA |
| 38 | gi\|4505904 | 5372 | Homo sapiens phosphomannomutase 1 (PMM1), mRNA |
| 39 | gi\|4506456 | 5955 | Homo sapiens reticulocalbin 2, EF-hand calcium binding domain (RCN2), mRNA |
| 40 | gi\|4506742 | 6202 | Homo sapiens ribosomal protein S8 (RPS8), mRNA |
| 41 | gi\|46198303 | 51287 | Homo sapiens coiled-coil-helix-coiled-coil-helix domain containing 8 (CHCHD8), mRNA |
| 42 | gi\|47078280 | 9679 | Homo sapiens family with sequence similarity 53, member 8 (FAM53B), mRNA |
| 43 | gi\|47132573 | 5571 | Homo sapiens protein kinase, AMP-activated, gamma 1 non-catalytic subunit (PRKAG1), |
| | | | transcript variant 1, mRNA |
| 44 | gi\|48375183 | 3476 | Homo sapiens immunoglobulin (CD79A) binding protein 1 (IGBP1), mRNA |
| 45 | gi\|51465675 | | Homo sapiens chromosome 6 genomic contig, reference assembly |
| 46 | gi\|52856410 | 30827 | Homo sapiens CXXC finger 1 (PHD domain) (CXXC1), mRNA |
| 47 | gi\|53759121 | 324 | Homo sapiens adenomatosis polyposis coli (APC), mRNA |
| 48 | gi\|55925645 | 27101 | Homo sapiens calcyclin binding protein (CACYBP), transcript variant 1, mRNA |
| 49 | gi\|56676339 | 51710 | Homo sapiens zinc finger protein 44 (ZNF44), mRNA |
| 50 | gi\|56790944 | 5499 | Homo sapiens protein phosphatase 1, catalytic subunit, alpha isoform (PPP1CA), transcript |
| | | | variant 3, mRNA |
| 51 | gi\|57242773 | 9703 | Homo sapiens KIAA0100 (KIAA0100), mRNA |
| 52 | gi\|57617038 | 23170 | Homo sapiens tubulin tyrosine ligase-like family, member 12 (TTLL12), mRNA |
| 53 | gi\|62953138 | 57727 | Homo sapiens nuclear receptor coactivator 5 (NCOA5), mRNA |
| 54 | gi\|63082031 | 23113 | Homo sapiens p53-associated parkin-like cytoplasmic protein (PARC), mRNA |
| 55 | gi\|65301138 | 10079 | Homo sapiens ATPase, Class II, type 9A (ATP9A), mRNA |
| 56 | gi\|68448524 | 972 | Homo sapiens CD74 molecule, major histocompatibility complex, class II invariant chain (CD74), |
| | | | transcript variant 2, mRNA |
| 57 | gi\|74048536 | 64219 | Homo sapiens praja 1 (PJA1), transcript variant 2, mRNA |
| 58 | gi\|7705609 | 51012 | Homo sapiens chromosome 20 open reading frame 45 (C20orf45), mRNA |
| 59 | gi\|7706556 | 51759 | Homo sapiens chromosome 9 open reading frame 78 (C9orf78), transcript variant 2, mRNA |
| 60 | gi\|77628146 | 10961 | Homo sapiens endoplasmic reticulum protein 29 (ERP29), transcript variant 1, mRNA |
| 61 | gi\|86788141 | 9844 | Homo sapiens engulfment and cell motility 1 (ELMO1), transcript variant 2, mRNA |
| 62 | gi\|88942921 | | Homo sapiens chromosome 1 genomic contig, reference assembly |
| 63 | gi\|88979823 | | Homo sapiens chromosome 4 genomic contig, alternate assembly (based on Celera assembly) |
| 64 | gi\|92859637 | 6861 | Homo sapiens synaptotagmin V (SYT5), mRNA |
| 65 | gi\|113412087 | 9672 | PREDICTED: Homo sapiens syndecan 3 (N-syndecan), transcript variant 2 (SDC3), mRNA |
| 66 | gi\|142385096 | 641700 | Homo sapiens endothelial cell-specific chemotaxis regulator (ECSCR), mRNA |
| 67 | gi\|222144282 | 65998 | Homo sapiens hypothetical protein LOC65998 (LOC65998), mRNA |
| 68 | gi\|113423046 | 26011 | Homo sapiens odz, odd Oz/ten-m homolog 4 (Drosophila) (ODZ4), mRNA |
| 69 | gi\|169790824 | 283450 | Homo sapiens chromosome 12 open reading frame 51 (C12orf51), mRNA |
| 70 | gi\|111607440 | 400506 | Homo sapiens chromosome 16 open reading frame 88 (C16orf88), mRNA |
| 71 | gi\|150010638 | 23326 | Homo sapiens ubiquitin specific peptidase 22 (USP22), mRNA |
| 72 | gi\|148839304 | 23347 | Homo sapiens structural maintenance of chromosomes flexible hinge domain containing 1 |
| | | | (SMCHD1), mRNA |
| 73 | gi\|113428453 | 57662 | Homo sapiens KIAA1543 (KIAA1543), mRNA |
| 74 | gi\|113429692 | 389906 | PREDICTED: Homo sapiens similar to Serine/threonine-protein kinase PRKX (Protein kinase |
| | | | PKX1) (LOC389906), mRNA |
| 75 | gi\|11386142 | 5345 | Homo sapiens serpin peptidase inhibitor, clade F (alpha-2 antiplasmin, pigment epithelium |
| | | | derived factor), member 2 (SERPINF2), mRNA |
| 76 | gi\|12056481 | 9310 | Homo sapiens zinc finger protein 235 (ZNF235), mRNA |
| 77 | gi\|12408642 | 8019 | Homo sapiens bromodomain containing 3 (BRD3), mRNA |
| 78 | gi\|12408650 | 7520 | Homo sapiens X-ray repair complementing defective repair in Chinese hamster cells 5 (double- |
| | | | strand-break rejoining; Ku autoantigen, 80 kDa) (XRCC5), mRNA |
| 79 | gi\|12545399 | 3385 | Homo sapiens intercellular adhesion molecule 3 (ICAM3), mRNA |
| 80 | gi\|13112047 | 2261 | Homo sapiens fibroblast growth factor receptor 3 (achondroplasia, thanatophoric dwarfism) |
| | | | (FGFR3), transcript variant 2, mRNA |
| 81 | gi\|13236584 | 79187 | Homo sapiens fibronectin type III and SPRY domain containing 1 (FSD1), mRNA |
| 82 | gi\|13236586 | 79188 | Homo sapiens transmembrane protein 43 (TMEM43), mRNA |
| 83 | gi\|13376149 | 79844 | Homo sapiens zinc finger, DHHC-type containing 11 (ZDHHC11), mRNA |
| 84 | gi\|13376239 | 79891 | Homo sapiens zinc finger protein 671 (ZNF671), mRNA |
| 85 | gi\|13376368 | 79969 | Homo sapiens chromosome 6 open reading frame 134 (C6orf134), transcript variant 2, mRNA |
| 86 | gi\|13435352 | 3897 | Homo sapiens L1 cell adhesion molecule (L1CAM), transcript variant 2, mRNA |
| 87 | gi\|13443001 | 80154 | Homo sapiens hypothetical protein FLJ22795 (FLJ22795), mRNA |
| 88 | gi\|13514826 | 1655 | Homo sapiens DEAD (Asp-Glu-Ala-Asp) box polypeptide 5 (DDX5), mRNA |
| 89 | gi\|13994299 | 83862 | Homo sapiens transmembrane protein induced by tumor necrosis factor alpha (TMPIT), mRNA |
| 90 | gi\|14043021 | 4141 | Homo sapiens methionine-tRNA synthetase (MARS), mRNA |
| 91 | gi\|14195613 | 56129 | Homo sapiens protocadherin beta 7 (PCDHB7), mRNA |
| 92 | gi\|14196449 | 56105 | Homo sapiens protocadherin gamma subfamily A, 11 (PCDHGA11), transcript variant 1, mRNA |
| 93 | gi\|14211539 | 4343 | Homo sapiens Mov10, Moloney leukemia virus 10, homolog (mouse) (MOV10), mRNA |
| 94 | gi\|14249707 | 85007 | Homo sapiens alanine-glyoxylate aminotransferase 2-like 2 (AGXT2L2), transcript variant 1, |
| | | | mRNA |
| 95 | gi\|14589925 | 51294 | Homo sapiens protocadherin 12 (PCDH12), mRNA |
| 96 | gi\|14670372 | 57060 | Homo sapiens poly(rC) binding protein 4 (PCBP4), transcript variant 4, mRNA |
| 97 | gi\|14971416 | 10155 | Homo sapiens tripartite motif-containing 28 (TRIM28), mRNA |
| 98 | gi\|15718685 | 6134 | Homo sapiens ribosomal protein L10 (RPL10), mRNA |
| 99 | gi\|15721936 | 64951 | Homo sapiens mitochondrial ribosomal protein S24 (MRPS24), nuclear gene encoding |
| | | | mitochondrial protein, mRNA |
| 100 | gi\|15812219 | 6160 | Homo sapiens ribosomal protein L31 (RPL31), mRNA |
| 101 | gi\|16507965 | 2023 | Homo sapiens enolase 1, (alpha) (ENO1), mRNA |
| 102 | gi\|16950626 | 1174 | Homo sapiens adaptor-related protein complex 1, sigma 1 subunit (AP1S1), transcript variant 1, |
| | | | mRNA |
| 103 | gi\|16950656 | 894 | Homo sapiens cyclin D2 (CCND2), mRNA |
| 104 | gi\|16950659 | 1022 | Homo sapiens cyclin-dependent kinase 7 (MO15 homolog, Xenopus laevis, cdk-activating kinase) |
| | | | (CDK7), mRNA |
| 105 | gi\|17149812 | 116138 | Homo sapiens kelch domain containing 3 (KLHDC3), mRNA |
| 106 | gi\|17388802 | 10606 | Homo sapiens phosphoribosylaminoimidazole carboxylase, phosphoribosylaminoimidazole |
| | | | succinocarboxamide synthetase (PAICS), mRNA |
| 107 | gi\|17505192 | 7477 | Homo sapiens wingless-type MMTV integration site family, member 7B (WNT7B), mRNA |
| 108 | gi\|17530790 | 117608 | Homo sapiens zinc finger protein 354B (ZNF354B), mRNA |
| 109 | gi\|17978480 | 64601 | Homo sapiens vacuolar protein sorting 16 (yeast) (VPS16), transcript variant 3, mRNA |
| 110 | gi\|17978511 | 22827 | Homo sapiens fuse-binding protein-interacting repressor (SIAHBP1), transcript variant 1, mRNA |
| 111 | gi\|18105005 | 8907 | Homo sapiens adaptor-related protein complex 1, mu 1 subunit (AP1M1), mRNA |
| 112 | gi\|18105057 | 26276 | Homo sapiens vacuolar protein sorting 33B (yeast) (VPS33B), mRNA |
| 113 | gi\|18201904 | 2821 | Homo sapiens glucose phosphate isomerase (GPI), mRNA |
| 114 | gi\|18375629 | 7917 | Homo sapiens HLA-B associated transcript 3 (BAT3), transcript variant 2, mRNA |
| 115 | gi\|18426914 | 1627 | Homo sapiens drebrin 1 (DBN1), transcript variant 1, mRNA |
| 116 | gi\|19263342 | 119016 | Homo sapiens centaurin, gamma-like family, member 1 (CTGLF1), mRNA |
| 117 | gi\|19913344 | 7884 | Homo sapiens stem-loop (histone) binding protein (SLBP), mRNA |
| 118 | gi\|19923783 | 10923 | Homo sapiens SUB1 homolog (S. cerevisiae) (SUB1), mRNA |
| 119 | gi\|20127439 | 10227 | Homo sapiens tetracycline transporter-like protein (TETRAN), mRNA |
| 120 | gi\|20127622 | 79084 | Homo sapiens WD repeat domain 77 (WDR77), mRNA |
| 121 | gi\|20270302 | 89941 | Homo sapiens ras homolog gene family, member T2 (RHOT2), mRNA |
| 122 | gi\|20270356 | 130617 | Homo sapiens zinc finger, AN1-type domain 2B (ZFAND2B), mRNA |
| 123 | gi\|20336204 | 546 | Homo sapiens alpha thalassemia/mental retardation syndrome X-linked (RAD54 homolog, S. |
| | | | cerevisiae) (ATRX), transcript variant 2, mRNA |
| 124 | gi\|20357555 | 2017 | Homo sapiens cortactin (CTTN), transcript variant 2, mRNA |
| 125 | gi\|20558635 | | Homo sapiens chromosome 20 genomic contig, reference assembly |
| 126 | gi\|20986498 | 5598 | Homo sapiens mitogen-activated protein kinase 7 (MAPK7), transcript variant 2, mRNA |
| 127 | gi\|20986517 | 9479 | Homo sapiens mitogen-activated protein kinase 8 interacting protein 1 (MAPK8IP1), mRNA |
| 128 | gi\|21040276 | 221955 | Homo sapiens diacylglycerol lipase beta (DAGLBETA), mRNA |
| 129 | gi\|21070955 | 25839 | Homo sapiens component of oligomeric golgi complex 4 (COG4), mRNA |
| 130 | gi\|21070996 | 6786 | Homo sapiens stromal interaction molecule 1 (STIM1), mRNA |
| 131 | gi\|21265107 | 7103 | Homo sapiens tetraspanin 8 (TSPAN8), mRNA |
| 132 | gi\|21361550 | 6522 | Homo sapiens solute carrier family 4, anion exchanger, member 2 (erythrocyte membrane protein |
| | | | band 3-like 1) (SLC4A2), mRNA |
| 133 | gi\|21361610 | 51177 | Homo sapiens pleckstrin homology domain containing, family O member 1 (PLEKHO1), mRNA |
| 134 | gi\|21361948 | 81926 | Homo sapiens family with sequence similarity 108, member A1 (FAM108A1), mRNA |
| 135 | gi\|21362049 | 84317 | Homo sapiens coiled-coil domain containing 115 (CCDC115), mRNA |
| 136 | gi\|21389334 | 90809 | Homo sapiens transmembrane protein 55B (TMEM55B), mRNA |
| 137 | gi\|21396488 | 9361 | Homo sapiens Ion peptidase 1, mitochondrial (LONP1), nuclear gene encoding mitochondrial |
| | | | protein, mRNA |
| 138 | gi\|21536463 | 4356 | Homo sapiens membrane protein, palmitoylated 3 (MAGUK p55 subfamily member 3) (MPP3), |
| | | | mRNA |
| 139 | gi\|21687111 | 112476 | Homo sapiens proline-rich transmembrane protein 2 (PRRT2), mRNA |
| 140 | gi\|21735549 | 4294 | Homo sapiens mitogen-activated protein kinase kinase kinase 10 (MAP3K10), mRNA |
| 141 | gi\|21735623 | 7534 | Homo sapiens tyrosine 3-monooxygenase/tryptophan 5-monooxygenase activation protein, zeta |
| | | | polypeptide (YWHAZ), transcript variant 1, mRNA |
| 142 | gi\|21735624 | 7534 | Homo sapiens tyrosine 3-monooxygenase/tryptophan 5-monooxygenase activation protein, zeta |
| | | | polypeptide (YWHAZ), transcript variant 2, mRNA |
| 143 | gi\|22035552 | 322 | Homo sapiens amyloid beta (A4) precursor protein-binding, family B, member 1 (Fe65) (APBB1), |
| | | | transcript variant 1, mRNA |
| 144 | gi\|22035553 | 322 | Homo sapiens amyloid beta (A4) precursor protein-binding, family B, member 1 (Fe65) (APBB1), |
| | | | transcript variant 2, mRNA |
| 145 | gi\|22035561 | 55290 | Homo sapiens BRF2, subunit of RNA polymerase III transcription initiation factor, BRF1-like |
| | | | (BRF2), mRNA |
| 146 | gi\|22035607 | 114879 | Homo sapiens oxysterol binding protein-like 5 (OSBPL5), transcript variant 1, mRNA |
| 147 | gi\|22095338 | 5230 | Homo sapiens phosphoglycerate kinase 1 (PGK1), mRNA |
| 148 | gi\|22095346 | 23705 | Homo sapiens immunoglobulin superfamily, member 4 (IGSF4), mRNA |
| 149 | gi\|22095348 | 23038 | Homo sapiens WD and tetratricopeptide repeats 1 (WDTC1), mRNA |
| 150 | gi\|22095350 | 23039 | Homo sapiens exportin 7 (XPO7), mRNA |
| 151 | gi\|22538463 | 5690 | Homo sapiens proteasome (prosome, macropain) subunit, beta type, 2 (PSMB2), mRNA |
| 152 | gi\|22547145 | 55336 | Homo sapiens F-box and leucine-rich repeat protein 8 (FBXL8), mRNA |
| 153 | gi\|22547185 | 6470 | Homo sapiens serine hydroxymethyltransferase 1 (soluble) (SHMT1), transcript variant 1, mRNA |
| 154 | gi\|22748758 | 125144 | Homo sapiens chromosome 17 open reading frame 45 (C17orf45), mRNA |
| 155 | gi\|22748780 | 126282 | Homo sapiens tumor necrosis factor, alpha-induced protein 8-like 1 (TNFAIP8L1), mRNA |
| 156 | gi\|22907051 | 10552 | Homo sapiens actin related protein 2/3 complex, subunit 1A, 41 kDa (ARPC1A), mRNA |
| 157 | gi\|23111044 | 8723 | Homo sapiens sorting nexin 4 (SNX4), mRNA |
| 158 | gi\|23199996 | 55695 | Homo sapiens NOL1/NOP2/Sun domain family, member 5 (NSUN5), transcript variant 2, mRNA |
| 159 | gi\|23308571 | 79143 | Homo sapiens leukocyte receptor cluster (LRC) member 4 (LENG4), mRNA |
| 160 | gi\|23510380 | 3842 | Homo sapiens transportin 1 (TNPO1), transcript variant 2, mRNA |
| 161 | gi\|23510447 | 4174 | Homo sapiens MCM5 minichromosome maintenance deficient 5, cell division cycle 48 (S. |
| | | | cerevisiae) (MCM5), mRNA |
| 162 | gi\|23510450 | 327 | Homo sapiens N-acylaminoacyl-peptide hydrolase (APEH), mRNA |
| 163 | gi\|23943927 | 140680 | Homo sapiens chromosome 20 open reading frame 96 (C20orf96), mRNA |
| 164 | gi\|24307888 | 9100 | Homo sapiens ubiquitin specific peptidase 10 (USP10), mRNA |
| 165 | gi\|24308188 | 56963 | Homo sapiens RGM domain family, member A (RGMA), mRNA |
| 166 | gi\|24308325 | 84446 | Homo sapiens BR serine/threonine kinase 1 (BRSK1), mRNA |
| 167 | gi\|24308431 | 91875 | Homo sapiens tetratricopeptide repeat domain 5 (TTC5), mRNA |
| 168 | gi\|24430150 | 5700 | Homo sapiens proteasome (prosome, macropain) 26S subunit, ATPase, 1 (PSMC1), mRNA |
| 169 | gi\|24431972 | 54862 | Homo sapiens coiled-coil and C2 domain containing 1A (CC2D1A), mRNA |
| 170 | gi\|24431995 | 56904 | Homo sapiens SH3-domain GRB2-like endophilin B2 (SH3GLB2), mRNA |
| 171 | gi\|24432025 | 84861 | Homo sapiens kelch-like 22 (Drosophila) (KLHL22), mRNA |
| 172 | gi\|24476015 | 23432 | Homo sapiens G protein-coupled receptor 161 (GPR161), transcript variant 2, mRNA |
| 173 | gi\|25306276 | 85476 | Homo sapiens G elongation factor, mitochondrial 1 (GFM1), nuclear gene encoding mitochondrial |
| | | | protein, mRNA |
| 174 | gi\|25777644 | 3781 | Homo sapiens potassium intermediate/small conductance calcium-activated channel, subfamily |
| | | | N, member 2 (KCNN2), transcript variant 1, mRNA |
| 175 | gi\|25777676 | 9770 | Homo sapiens Ras association (RalGDS/AF-6) domain family 2 (RASSF2), transcript variant 2, |
| | | | mRNA |
| 176 | gi\|26667164 | 64978 | Homo sapiens mitochondrial ribosomal protein L38 (MRPL38), nuclear gene encoding |
| | | | mitochondrial protein, mRNA |
| 177 | gi\|27262633 | 4678 | Homo sapiens nuclear autoantigenic sperm protein (histone-binding) (NASP), transcript variant 1, |
| | | | mRNA |
| 178 | gi\|27374999 | 11345 | Homo sapiens GABA(A) receptor-associated protein-like 2 (GABARAPL2), mRNA |
| 179 | gi\|27477128 | 4157 | Homo sapiens melanocortin 1 receptor (alpha melanocyte stimulating hormone receptor) |
| | | | (MC1R), mRNA |
| 180 | gi\|28178837 | 3421 | Homo sapiens isocitrate dehydrogenase 3 (NAD+) gamma (IDH3G), nuclear gene encoding |
| | | | mitochondrial protein, transcript variant 2, mRNA |
| 181 | gi\|28416941 | 6632 | Homo sapiens small nuclear ribonucleoprotein D1 polypeptide 16 kDa (SNRPD1), mRNA |
| 182 | gi\|28872807 | 2596 | Homo sapiens growth associated protein 43 (GAP43), mRNA |
| 183 | gi\|28933450 | 23129 | Homo sapiens plexin D1 (PLXND1), mRNA |
| 184 | gi\|29171704 | 10916 | Homo sapiens melanoma antigen family D, 2 (MAGED2), transcript variant 2, mRNA |
| 185 | gi\|29740881 | | Homo sapiens chromosome 6 genomic contig, reference assembly |
| 186 | gi\|29791451 | | Homo sapiens chromosome 2 genomic contig, reference assembly |
| 187 | gi\|29791548 | | Homo sapiens chromosome 4 genomic contig, reference assembly |
| 188 | gi\|30179901 | 6659 | Homo sapiens SRY (sex determining region Y)-box 4 (SOX4), mRNA |
| 189 | gi\|30181235 | 221184 | Homo sapiens copine II (CPNE2), mRNA |
| 190 | gi\|31341385 | 5383 | Homo sapiens postmeiotic segregation increased 2-like 5 (PMS2L5), mRNA |
| 191 | gi\|31341899 | 196403 | Homo sapiens deltex 3 homolog (Drosophila) (DTX3), mRNA |
| 192 | gi\|31342615 | 337867 | Homo sapiens phosphoglycerate dehydrogenase like 1 (PHGDHL1), mRNA |
| 193 | gi\|31343375 | 137984 | Homo sapiens 1-acylglycerol-3-phosphate O-acyltransferase 6 (lysophosphatidic acid |
| | | | acyltransferase, zeta) (AGPAT6), mRNA |
| 194 | gi\|31343395 | 201164 | Homo sapiens similar to CG12314 gene product (LOC201164), mRNA |
| 195 | gi\|31377649 | 84866 | Homo sapiens transmembrane protein 25 (TMEM25), mRNA |
| 196 | gi\|31377801 | 6405 | Homo sapiens sema domain, immunoglobulin domain (Ig), short basic domain, secreted, |
| | | | (semaphorin) 3F (SEMA3F), mRNA |
| 197 | gi\|31542734 | 79703 | Homo sapiens hypothetical protein FLJ22531 (FLJ22531), mRNA |
| 198 | gi\|31542944 | 51493 | Homo sapiens chromosome 22 open reading frame 28 (C22orf28), mRNA |
| 199 | gi\|31543803 | 7980 | Homo sapiens tissue factor pathway inhibitor 2 (TFPI2), mRNA |
| 200 | gi\|31652217 | 6837 | Homo sapiens surfeit 5 (SURF5), transcript variant c, mRNA |
| 201 | gi\|32967304 | 51004 | Homo sapiens coenzyme Q6 homolog, monooxygenase (S. cerevisiae) (COQ6), transcript |
| | | | variant 1, mRNA |
| 202 | gi\|33186926 | 152137 | Homo sapiens coiled-coil domain containing 50 (CCDC50), transcript variant 1, mRNA |
| 203 | gi\|33188442 | 23499 | Homo sapiens microtubule-actin crosslinking factor 1 (MACF1), transcript variant 2, mRNA |
| 204 | gi\|33356548 | 4172 | Homo sapiens MCM3 minichromosome maintenance deficient 3 (S. cerevisiae) (MCM3), mRNA |
| 205 | gi\|33359212 | 5310 | Homo sapiens polycystic kidney disease 1 (autosomal dominant) (PKD1), transcript variant 2, |
| | | | mRNA |
| 206 | gi\|33359689 | 1948 | Homo sapiens ephrin-B2 (EFNB2), mRNA |
| 207 | gi\|33413399 | 2098 | Homo sapiens esterase D/formylglutathione hydrolase (ESD), mRNA |
| 208 | gi\|33466360 | 10139 | Homo sapiens ADP-ribosylation factor related protein 1 (ARFRP1), mRNA |
| 209 | gi\|33519462 | 4705 | Homo sapiens NADH dehydrogenase (ubiquinone) 1 alpha subcomplex, 10, 42 kDa (NDUFA10), |
| | | | nuclear gene encoding mitochondrial protein, mRNA |
| 210 | gi\|33598938 | 91179 | Homo sapiens scavenger receptor class F, member 2 (SCARF2), transcript variant 1, mRNA |
| 211 | gi\|33636749 | 63893 | Homo sapiens ubiquitin-conjugating enzyme E2O (UBE2O), mRNA |
| 212 | gi\|34147352 | 79002 | Homo sapiens chromosome 19 open reading frame 43 (C19orf43), mRNA |
| 213 | gi\|34147392 | 79363 | Homo sapiens chromosome 1 open reading frame 89 (C1orf89), mRNA |
| 214 | gi\|34147499 | 5504 | Homo sapiens protein phosphatase 1, regulatory (inhibitor) subunit 2 (PPP1R2), mRNA |
| 215 | gi\|34147502 | 58491 | Homo sapiens zinc finger protein 71 (ZNF71), mRNA |
| 216 | gi\|34147585 | 51433 | Homo sapiens anaphase promoting complex subunit 5 (ANAPC5), mRNA |
| 217 | gi\|34147615 | 83723 | Homo sapiens family with sequence similarity 57, member B (FAM57B), mRNA |
| 218 | gi\|34147632 | 5347 | Homo sapiens polo-like kinase 1 (Drosophila) (PLK1), mRNA |
| 219 | gi\|34147640 | 60528 | Homo sapiens elaC homolog 2 (E. coli) (ELAC2), mRNA |
| 220 | gi\|34147695 | 84946 | Homo sapiens LTV1 homolog (S. cerevisiae) (LTV1), mRNA |
| 221 | gi\|34147717 | 115704 | Homo sapiens ecotropic viral integration site 5-like (EVI5L), mRNA |
| 222 | gi\|34222164 | 91392 | Homo sapiens zinc finger protein 502 (ZNF502), mRNA |
| 223 | gi\|34222207 | 146330 | Homo sapiens F-box and leucine-rich repeat protein 16 (F8XL16), mRNA |
| 224 | gi\|34222313 | 27339 | Homo sapiens PRP19/PSO4 pre-mRNA processing factor 19 homolog (S. cerevisiae) (PRPF19), |
| | | | mRNA |
| 225 | gi\|34222340 | 80148 | Homo sapiens PQ loop repeat containing 1 (PQLC1), mRNA |
| 226 | gi\|34222352 | 79751 | Homo sapiens solute carrier family 25 (mitochondrial carrier: glutamate), member 22 |
| | | | (SLC25A22), mRNA |
| 227 | gi\|34303923 | 147923 | Homo sapiens zinc finger protein 420 (ZNF420), mRNA |
| 228 | gi\|34328935 | 6634 | Homo sapiens small nuclear ribonucleoprotein D3 polypeptide 18 kDa (SNRPD3), mRNA |
| 229 | gi\|34335143 | 6166 | Homo sapiens ribosomal protein L36a-like (RPL36AL), mRNA |
| 230 | gi\|34335149 | 6205 | Homo sapiens ribosomal protein S11 (RPS11), mRNA |
| 231 | gi\|34335279 | 5713 | Homo sapiens proteasome (prosome, macropain) 26S subunit, non-ATPase, 7 (Mov34 homolog) |
| | | | (PSMD7), mRNA |
| 232 | gi\|34335395 | 666 | Homo sapiens BCL2-related ovarian killer (BOK), mRNA |
| 233 | gi\|34452691 | 51160 | Homo sapiens vacuolar protein sorting 28 homolog (S. cerevisiae) (VPS28), transcript variant 1, |
| | | | mRNA |
| 234 | gi\|34577108 | 226 | Homo sapiens aldolase A, fructose-bisphosphate (ALDOA), transcript variant 1, mRNA |
| 235 | gi\|36287068 | 10524 | Homo sapiens HIV-1 Tat interacting protein, 60 kDa (HTATIP), transcript variant 1, mRNA |
| 236 | gi\|36287129 | 64432 | Homo sapiens mitochondrial ribosomal protein S25 (MRPS25), nuclear gene encoding |
| | | | mitochondrial protein, mRNA |
| 237 | gi\|37059733 | 3030 | Homo sapiens hydroxyacyl-Coenzyme A dehydrogenase/3-ketoacyl-Coenzyme A thiolase/enoyl- |
| | | | Coenzyme A hydratase (trifunctional protein), alpha subunit (HADHA), mRNA |
| 238 | gi\|37059754 | 90353 | Homo sapiens ATP binding domain 3 (ATPBD3), mRNA |
| 239 | gi\|37059801 | 51239 | Homo sapiens ankyrin repeat domain 39 (ANKRD39), mRNA |
| 240 | gi\|37537720 | 126792 | Homo sapiens UDP-Gal: betaGal beta 1,3-galactosyltransferase polypeptide 6 (B3GALT6), mRNA |
| 241 | gi\|37544588 | | Homo sapiens chromosome 17 genomic contig, reference assembly |
| 242 | gi\|37546841 | | Homo sapiens chromosome 13 genomic contig, reference assembly |
| 243 | gi\|37574605 | 7750 | Homo sapiens zinc finger, MYM-type 2 (ZMYM2), mRNA |
| 244 | gi\|37574721 | | Homo sapiens chromosome 19 genomic contig, reference assembly |
| 245 | gi\|37594439 | 10467 | Homo sapiens zinc finger, HIT type 1 (ZNHIT1), mRNA |
| 246 | gi\|38016912 | 8683 | Homo sapiens splicing factor, arginine/serine-rich 9 (SFRS9), mRNA |
| 247 | gi\|38146101 | 27343 | Homo sapiens polymerase (DNA directed), lambda (POLL), mRNA |
| 248 | gi\|38176297 | 10715 | Homo sapiens LAG1 longevity assurance homolog 1 (S. cerevisiae) (LASS1), transcript variant 1, |
| | | | mRNA |
| 249 | gi\|38201654 | 54556 | Homo sapiens inhibitor of growth family, member 3 (ING3), transcript variant 1, mRNA |
| 250 | gi\|38488711 | 128989 | Homo sapiens chromosome 22 open reading frame 25 (C22orf25), mRNA |
| 251 | gi\|38569402 | 403 | Homo sapiens ADP-ribosylation factor-like 3 (ARL3), mRNA |
| 252 | gi\|38570153 | 23567 | Homo sapiens zinc finger protein 346 (ZNF346), mRNA |
| 253 | gi\|38570154 | 64101 | Homo sapiens leucine rich repeat containing 4 (LRRC4), mRNA |
| 254 | gi\|38683847 | 9158 | Homo sapiens fibroblast growth factor (acidic) intracellular binding protein (FIBP), transcript |
| | | | variant 2, mRNA |
| 255 | gi\|38788218 | 126299 | Homo sapiens zinc finger protein 428 (ZNF428), mRNA |
| 256 | gi\|39725639 | 54973 | Homo sapiens cleavage and polyadenylation specific factor 3-like (CPSF3L), transcript variant 1, |
| | | | mRNA |
| 257 | gi\|39725712 | 5213 | Homo sapiens phosphofructokinase, muscle (PFKM), mRNA |
| 258 | gi\|39725941 | 56652 | Homo sapiens progressive external ophthalmoplegia 1 (PEO1), mRNA |
| 259 | gi\|39811997 | 166 | Homo sapiens amino-terminal enhancer of split (AES), transcript variant 2, mRNA |
| 260 | gi\|40068460 | 11344 | Homo sapiens PTK9L protein tyrosine kinase 9-like (A6-related protein) (PTK9L), mRNA |
| 261 | gi\|40217846 | 23020 | Homo sapiens activating signal cointegrator 1 complex subunit 3-like 1 (ASCC3L1), mRNA |
| 262 | gi\|40254856 | 22846 | Homo sapiens vasohibin 1 (VASH1), mRNA |
| 263 | gi\|40254860 | 9898 | Homo sapiens ubiquitin associated protein 2-like (UBAP2L), mRNA |
| 264 | gi\|40254865 | 25978 | Homo sapiens chromatin modifying protein 2B (CHMP2B), mRNA |
| 265 | gi\|40255056 | 116372 | Homo sapiens LY6/PLAUR domain containing 1 (LYPD1), mRNA |
| 266 | gi\|40255100 | 91252 | Homo sapiens solute carrier family 39 (zinc transporter), member 13 (SLC39A13), mRNA |
| 267 | gi\|40288188 | 64236 | Homo sapiens PDZ and LIM domain 2 (mystique) (PDLIM2), transcript variant 2, mRNA |
| 268 | gi\|40353202 | 57184 | Homo sapiens chromosome 15 open reading frame 17 (C15orf17), mRNA |
| 269 | gi\|40806168 | 6683 | Homo sapiens spastin (SPAST), transcript variant 1, mRNA |
| 270 | gi\|41281370 | 2224 | Homo sapiens farnesyl diphosphate synthase (farnesyl pyrophosphate synthetase, |
| | | | dimethylallyltranstransferase, geranyltranstransferase) (FDPS), mRNA |
| 271 | gi\|41281380 | 9902 | Homo sapiens mannose receptor, C type 2 (MRC2), mRNA |
| 272 | gi\|41281440 | 9726 | Homo sapiens zinc finger protein 646 (ZNF646), mRNA |
| 273 | gi\|41281446 | 9742 | Homo sapiens intraflagellar transport 140 homolog (Chlamydomonas) (IFT140), mRNA |
| 274 | gi\|41281472 | 9772 | Homo sapiens KIAA0195 (KIAA0195), mRNA |
| 275 | gi\|41327758 | 539 | Homo sapiens ATP synthase, H+ transporting, mitochondrial F1 complex, O subunit (oligomycin |
| | | | sensitivity conferring protein) (ATP5O), nuclear gene encoding mitochondrial protein, mRNA |
| 276 | gi\|41327778 | 57696 | Homo sapiens DEAD (Asp-Glu-Ala-Asp) box polypeptide 55 (DDX55), mRNA |
| 277 | gi\|41327780 | 25915 | Homo sapiens chromosome 3 open reading frame 60 (C3orf60), transcript variant 1, mRNA |
| 278 | gi\|41352715 | 8568 | Homo sapiens DNA segment on chromosome 21 (unique) 2056 expressed sequence |
| | | | (D21S2056E), mRNA |
| 279 | gi\|41352716 | 51366 | Homo sapiens E3 ubiquitin protein ligase, HECT domain containing, 1 (EDD1), mRNA |
| 280 | gi\|41393546 | 51594 | Homo sapiens neuroblastoma-amplified protein (NAG), mRNA |
| 281 | gi\|41872373 | 1263 | Homo sapiens polo-like kinase 3 (Drosophila) (PLK3), mRNA |
| 282 | gi\|41872408 | 5473 | Homo sapiens pro-platelet basic protein (chemokine (C-X-C motif) ligand 7) (PPBP), mRNA |
| 283 | gi\|41872442 | 26133 | Homo sapiens transient receptor potential cation channel, subfamily C, member 4 associated |
| | | | protein (TRPC4AP), transcript variant 2, mRNA |
| 284 | gi\|42415528 | 6508 | Homo sapiens solute carrier family 4, anion exchanger, member 3 (SLC4A3), transcript variant 2, |
| | | | mRNA |
| 285 | gi\|42476205 | 51308 | Homo sapiens receptor accessory protein 2 (REEP2), mRNA |
| 286 | gi\|42544225 | 57617 | Homo sapiens vacuolar protein sorting protein 18 (VPS18), mRNA |
| 287 | gi\|42544228 | 7186 | Homo sapiens TNF receptor-associated factor 2 (TRAF2), mRNA |
| 288 | gi\|42544230 | 10446 | Homo sapiens leucine rich repeat neuronal 5 (LRRN5), transcript variant 1, mRNA |
| 289 | gi\|42734423 | 10213 | Homo sapiens proteasome (prosome, macropain) 26S subunit, non-ATPase, 14 (PSMD14), |
| | | | mRNA |
| 290 | gi\|42734433 | 79639 | Homo sapiens transmembrane protein 53 (TMEM53), mRNA |
| 291 | gi\|44888817 | 22982 | Homo sapiens DIP2 disco-interacting protein 2 homolog C (Drosophila) (DIP2C), mRNA |
| 292 | gi\|44890060 | 5479 | Homo sapiens peptidylprolyl isomerase B (cyclophilin B) (PPIB), mRNA |
| 293 | gi\|44955913 | 9170 | Homo sapiens endothelial differentiation, lysophosphatidic acid G-protein-coupled receptor, 4 |
| | | | (EDG4), mRNA |
| 294 | gi\|4502368 | 8424 | Homo sapiens butyrobetaine (gamma), 2-oxoglutarate dioxygenase (gamma-butyrobetaine |
| | | | hydroxylase) 1 (BBOX1), mRNA |
| 295 | gi\|4502884 | 1198 | Homo sapiens CDC-like kinase 3 (CLK3), transcript variant phclk3, mRNA |
| 296 | gi\|4503970 | 2664 | Homo sapiens GDP dissociation inhibitor 1 (GDI1), mRNA |
| 297 | gi\|4505650 | 5833 | Homo sapiens phosphate cytidylyltransferase 2, ethanolamine (PCYT2), mRNA |
| 298 | gi\|4505940 | 5431 | Homo sapiens polymerase (RNA) II (DNA directed) polypeptide B, 140 kDa (POLR2B), mRNA |
| 299 | gi\|4506138 | 5119 | Homo sapiens procollagen (type III) N-endopeptidase (PCOLN3), mRNA |
| 300 | gi\|4506756 | 6262 | Homo sapiens ryanodine receptor 2 (cardiac) (RYR2), mRNA |
| 301 | gi\|4506890 | 6418 | Homo sapiens SET translocation (myeloid leukemia-associated) (SET), mRNA |
| 302 | gi\|4507520 | 7086 | Homo sapiens transketolase (Wernicke-Korsakoff syndrome) (TKT), mRNA |
| 303 | gi\|4507668 | 7178 | Homo sapiens tumor protein, translationally-controlled 1 (TPT1), mRNA |
| 304 | gi\|45433546 | 57513 | Homo sapiens CASK interacting protein 2 (CASKIN2), mRNA |
| 305 | gi\|45439320 | 5481 | Homo sapiens peptidylprolyl isomerase D (cyclophilin D) (PPID), mRNA |
| 306 | gi\|4557618 | 2632 | Homo sapiens glucan (1,4-alpha-), branching enzyme 1 (glycogen branching enzyme, Andersen |
| | | | disease, glycogen storage disease type IV) (GBE1), mRNA |
| 307 | gi\|4557692 | 3795 | Homo sapiens ketohexokinase (fructokinase) (KHK), transcript variant a, mRNA |
| 308 | gi\|4557808 | 4942 | Homo sapiens ornithine aminotransferase (gyrate atrophy) (OAT), nuclear gene encoding |
| | | | mitochondrial protein, mRNA |
| 309 | gi\|45593139 | 54676 | Homo sapiens GTP binding protein 2 (GTPBP2), mRNA |
| 310 | gi\|46094059 | 27018 | Homo sapiens nerve growth factor receptor (TNFRSF16) associated protein 1 (NGFRAP1), |
| | | | transcript variant 2, mRNA |
| 311 | gi\|46195756 | 114826 | Homo sapiens SET and MYND domain containing 4 (SMYD4), mRNA |
| 312 | gi\|46255048 | 3275 | Homo sapiens protein arginine methyltransferase 2 (PRMT2), transcript variant 1, mRNA |
| 313 | gi\|46359074 | 23090 | Homo sapiens zinc finger protein 423 (ZNF423), mRNA |
| 314 | gi\|46488918 | 23245 | Homo sapiens astrotactin 2 (ASTN2), transcript variant 3, mRNA |
| 315 | gi\|46592990 | 56850 | Homo sapiens GRIP1 associated protein 1 (GRIPAP1), transcript variant 1, mRNA |
| 316 | gi\|46909599 | 8751 | Homo sapiens ADAM metallopeptidase domain 15 (metargidin) (ADAM15), transcript variant 6, |
| | | | mRNA |
| 317 | gi\|47078217 | 20 | Homo sapiens ATP-binding cassette, sub-family A (ABC1), member 2 (ABCA2), transcript variant |
| | | | 2, mRNA |
| 318 | gi\|47078257 | 8702 | Homo sapiens UDP-Gal: betaGlcNAc beta 1,4-galactosyltransferase, polypeptide 4 (B4GALT4), |
| | | | transcript variant 1, mRNA |
| 319 | gi\|47078275 | 1119 | Homo sapiens choline kinase alpha (CHKA), transcript variant 1, mRNA |
| 320 | gi\|47132610 | 23192 | Homo sapiens ATG4 autophagy related 4 homolog B (S. cerevisiae) (ATG4B), transcript variant |
| | | | 1, mRNA |
| 321 | gi\|47174863 | 56915 | Homo sapiens exosome component 5 (EXOSC5), mRNA |
| 322 | gi\|47271442 | 6427 | Homo sapiens splicing factor, arginine/serine-rich 2 (SFRS2), mRNA |
| 323 | gi\|4757805 | 9605 | Homo sapiens chromosome 16 open reading frame 7 (C16orf7), mRNA |
| 324 | gi\|47578121 | 51566 | Homo sapiens armadillo repeat containing, X-linked 3 (ARMCX3), transcript variant 3, mRNA |
| 325 | gi\|4758119 | 1611 | Homo sapiens death-associated protein (DAP), mRNA |
| 326 | gi\|4758559 | 9410 | Homo sapiens WD repeat domain 57 (U5 snRNP specific) (WDR57), mRNA |
| 327 | gi\|4758955 | 9256 | Homo sapiens benzodiazapine receptor (peripheral) associated protein 1 (BZRAP1), mRNA |
| 328 | gi\|48255932 | 3150 | Homo sapiens high-mobility group nucleosome binding domain 1 (HMGN1), mRNA |
| 329 | gi\|48255950 | 491 | Homo sapiens ATPase, Ca++ transporting, plasma membrane 2 (ATP2B2), transcript variant 1, |
| | | | mRNA |
| 330 | gi\|4826959 | 5859 | Homo sapiens glutaminyl-tRNA synthetase (QARS), mRNA |
| 331 | gi\|48762680 | 476 | Homo sapiens ATPase, Na+/K+ transporting, alpha 1 polypeptide (ATP1A1), transcript variant 1, |
| | | | mRNA |
| 332 | gi\|48762925 | 5822 | Homo sapiens PWP2 periodic tryptophan protein homolog (yeast) (PWP2H), mRNA |
| 333 | gi\|4885584 | 10055 | Homo sapiens SUMO-1 activating enzyme subunit 1 (SAE1), mRNA |
| 334 | gi\|48976060 | 80724 | Homo sapiens acyl-Coenzyme A dehydrogenase family, member 10 (ACAD10), mRNA |
| 335 | gi\|49472840 | 26993 | Homo sapiens A kinase (PRKA) anchor protein 8-like (AKAP8L), mRNA |
| 336 | gi\|49574501 | 51706 | Homo sapiens cytochrome b5 reductase 1 (CYB5R1), mRNA |
| 337 | gi\|5031892 | 10062 | Homo sapiens nuclear receptor subfamily 1, group H, member 3 (NR1H3), mRNA |
| 338 | gi\|50345980 | 498 | Homo sapiens ATP synthase, H+ transporting, mitochondrial F1 complex, alpha subunit 1, |
| | | | cardiac muscle (ATP5A1), nuclear gene encoding mitochondrial protein, transcript variant 2, |
| | | | mRNA |
| 339 | gi\|50346004 | 5211 | Homo sapiens phosphofructokinase, liver (PFKL), transcript variant 1, mRNA |
| 340 | gi\|50363222 | 79005 | Homo sapiens sodium channel modifier 1 (SCNM1), transcript variant 1, mRNA |
| 341 | gi\|50409737 | 51329 | Homo sapiens ADP-ribosylation-like factor 6 interacting protein 4 (ARL6IP4), transcript variant 3, |
| | | | mRNA |
| 342 | gi\|50726984 | 51466 | Homo sapiens Enah/Vasp-like (EVL), mRNA |
| 343 | gi\|50811881 | 2820 | Homo sapiens glycerol-3-phosphate dehydrogenase 2 (mitochondrial) (GPD2), mRNA |
| 344 | gi\|50811906 | 1808 | Homo sapiens dihydropyrimidinase-like 2 (DPYSL2), mRNA |
| 345 | gi\|51093838 | 51386 | Homo sapiens eukaryotic translation initiation factor 3, subunit 6 interacting protein (EIF3S6IP), |
| | | | mRNA |
| 346 | gi\|51317356 | 10226 | Homo sapiens mannose-6-phosphate receptor binding protein 1 (M6PRBP1), mRNA |
| 347 | gi\|51464027 | | Homo sapiens chromosome 3 genomic contig, reference assembly |
| 348 | gi\|51467962 | | Homo sapiens chromosome 10 genomic contig, reference assembly |
| 349 | gi\|51477712 | 11253 | Homo sapiens mannosidase, alpha, class 1B, member 1 (MAN1B1), mRNA |
| 350 | gi\|51702205 | 254428 | Homo sapiens solute carrier family 41, member 1 (SLC41A1), mRNA |
| 351 | gi\|5174456 | 10403 | Homo sapiens kinetochore associated 2 (KNTC2), mRNA |
| 352 | gi\|51944964 | 10550 | Homo sapiens ADP-ribosylation-like factor 6 interacting protein 5 (ARL6IP5), mRNA |
| 353 | gi\|51972237 | 440400 | Homo sapiens similar to DNA segment, Chr 11, Brigham & Womens Genetics 0434 expressed |
| | | | (MGC71993), mRNA |
| 354 | gi\|52145308 | 84894 | Homo sapiens leucine rich repeat neuronal 6A (LRRN6A), mRNA |
| 355 | gi\|52426770 | 3105 | Homo sapiens major histocompatibility complex, class I, A (HLA-A), mRNA |
| 356 | gi\|52486264 | 9240 | Homo sapiens paraneoplastic antigen MA1 (PNMA1), mRNA |
| 357 | gi\|52856412 | 51118 | Homo sapiens UTP11-like, U3 small nucleolar ribonucleoprotein, (yeast) (UTP11L), mRNA |
| 358 | gi\|53749664 | 7025 | Homo sapiens nuclear receptor subfamily 2, group F, member 1 (NR2F1), mRNA |
| 359 | gi\|53759067 | 92979 | Homo sapiens membrane-associated ring finger (C3HC4) 9 (MARCH9), mRNA |
| 360 | gi\|53832010 | 8912 | Homo sapiens calcium channel, voltage-dependent, alpha 1H subunit (CACNA1H), transcript |
| | | | variant 2, mRNA |
| 361 | gi\|54112408 | 56005 | Homo sapiens chromosome 19 open reading frame 10 (C19orf10), mRNA |
| 362 | gi\|5453564 | 10458 | Homo sapiens BAI1-associated protein 2 (BAIAP2), transcript variant 3, mRNA |
| 363 | gi\|5453765 | 4753 | Homo sapiens NEL-like 2 (chicken) (NELL2), mRNA |
| 364 | gi\|5454057 | 6484 | Homo sapiens ST3 beta-galactoside alpha-2,3-sialyltransferase 4 (ST3GAL4), mRNA |
| 365 | gi\|5454161 | 10628 | Homo sapiens thioredoxin interacting protein (TXNIP), mRNA |
| 366 | gi\|54607107 | 83637 | Homo sapiens zinc finger, MIZ-type containing 2 (ZMIZ2), transcript variant 1, mRNA |
| 367 | gi\|55741674 | 22889 | Homo sapiens KIAA0907 (KIAA0907), mRNA |
| 368 | gi\|55741856 | 25981 | Homo sapiens dynein, axonemal, heavy chain 1 (DNAH1), mRNA |
| 369 | gi\|55742814 | 27154 | Homo sapiens bromodomain and PHD finger containing, 3 (BRPF3), mRNA |
| 370 | gi\|55749725 | 57542 | Homo sapiens kelch domain containing 5 (KLHDC5), mRNA |
| 371 | gi\|55749880 | 8562 | Homo sapiens density-regulated protein (DENR), mRNA |
| 372 | gi\|55749916 | 9681 | Homo sapiens DEP domain containing 5 (DEPDC5), transcript variant 1, mRNA |
| 373 | gi\|55769569 | 84911 | Homo sapiens zinc finger protein 382 (ZNF382), mRNA |
| 374 | gi\|55925575 | 3485 | Homo sapiens insulin-like growth factor binding protein 2, 36 kDa (IGFBP2), mRNA |
| 375 | gi\|55925651 | 51186 | Homo sapiens WW domain binding protein 5 (WBP5), transcript variant 1, mRNA |
| 376 | gi\|55956785 | 9814 | Homo sapiens Sfi1 homolog, spindle assembly associated (yeast) (SFI1), transcript variant 1, |
| | | | mRNA |
| 377 | gi\|56676386 | 23423 | Homo sapiens transmembrane emp24 protein transport domain containing 3 (TMED3), mRNA |
| 378 | gi\|56699487 | 9612 | Homo sapiens nuclear receptor co-repressor 2 (NCOR2), mRNA |
| 379 | gi\|56788363 | 85378 | Homo sapiens tubulin, gamma complex associated protein 6 (TUBGCP6), transcript variant 1, |
| | | | mRNA |
| 380 | gi\|57242795 | 10765 | Homo sapiens jumonji, AT rich interactive domain 1B (JARID1B), mRNA |
| 381 | gi\|5729771 | 1203 | Homo sapiens ceroid-lipofuscinosis, neuronal 5 (CLN5), mRNA |
| 382 | gi\|5730084 | 6993 | Homo sapiens dynein, light chain, Tctex-type 1 (DYNLT1), mRNA |
| 383 | gi\|57634538 | 6625 | Homo sapiens small nuclear ribonucleoprotein 70 kDa polypeptide (RNP antigen) (SNRP70), |
| | | | transcript variant 2, mRNA |
| 384 | gi\|57770467 | 146712 | Homo sapiens UDP-GlcNAc: betaGal beta-1,3-N-acetylglucosaminyltransferase-like 1 |
| | | | (B3GNTL1), mRNA |
| 385 | gi\|57863256 | 6950 | Homo sapiens t-complex 1 (TCP1), transcript variant 1, mRNA |
| 386 | gi\|57863280 | 56917 | Homo sapiens Meis1, myeloid ecotropic viral integration site 1 homolog 3 (mouse) (MEIS3), |
| | | | transcript variant 1, mRNA |
| 387 | gi\|57863294 | 23313 | Homo sapiens chromosome 22 open reading frame 9 (C22orf9), transcript variant 2, mRNA |
| 388 | gi\|57863311 | 54963 | Homo sapiens uridine-cytidine kinase 1-like 1 (UCKL1), mRNA |
| 389 | gi\|58331162 | 26147 | Homo sapiens PHD finger protein 19 (PHF19), transcript variant 2, mRNA |
| 390 | gi\|58331183 | 10574 | Homo sapiens chaperonin containing TCP1, subunit 7 (eta) (CCT7), transcript variant 1, mRNA |
| 391 | gi\|58331234 | 64411 | Homo sapiens centaurin, delta 3 (CENTD3), mRNA |
| 392 | gi\|58331236 | 55816 | Homo sapiens docking protein 5 (DOK5), mRNA |
| 393 | gi\|58530880 | 9077 | Homo sapiens DIRAS family, GTP-binding RAS-like 3 (DIRAS3), mRNA |
| 394 | gi\|58761485 | 7203 | Homo sapiens chaperonin containing TCP1, subunit 3 (gamma) (CCT3), transcript variant 2, |
| | | | mRNA |
| 395 | gi\|59889569 | 11051 | Homo sapiens nudix (nucleoside diphosphate linked moiety X)-type motif 21 (NUDT21), mRNA |
| 396 | gi\|6005965 | 7705 | Homo sapiens zinc finger protein 146 (ZNF146), mRNA |
| 397 | gi\|6005992 | 1211 | Homo sapiens clathrin, light polypeptide (Lca) (CLTA), transcript variant brain-specific, mRNA |
| 398 | gi\|60498972 | 23040 | Homo sapiens myelin transcription factor 1-like (MYT1L), mRNA |
| 399 | gi\|60499005 | 9570 | Homo sapiens golgi SNAP receptor complex member 2 (GOSR2), transcript variant A, mRNA |
| 400 | gi\|61744459 | 11091 | Homo sapiens WD repeat domain 5 (WDR5), transcript variant 1, mRNA |
| 401 | gi\|61966896 | 441208 | Homo sapiens hypothetical gene supported by AK094370 (LOC441208), mRNA |
| 402 | gi\|62240999 | 975 | Homo sapiens CD81 molecule (CD81), mRNA |
| 403 | gi\|62241007 | 1428 | Homo sapiens crystallin, mu (CRYM), transcript variant 2, mRNA |
| 404 | gi\|62241021 | 11269 | Homo sapiens DEAD (Asp-Glu-Ala-As) box polypeptide 19B (DDX19B), transcript variant 3, |
| | | | mRNA |
| 405 | gi\|62388887 | 3767 | Homo sapiens potassium inwardly-rectifying channel, subfamily J, member 11 (KCNJ11), mRNA |
| 406 | gi\|62388891 | 3838 | Homo sapiens karyopherin alpha 2 (RAG cohort 1, importin alpha 1) (KPNA2), mRNA |
| 407 | gi\|62530390 | 6414 | Homo sapiens selenoprotein P, plasma, 1 (SEPP1), mRNA |
| 408 | gi\|63079686 | 6468 | Homo sapiens F-box and WD-40 domain protein 4 (FBXW4), mRNA |
| 409 | gi\|63162571 | 7203 | Homo sapiens chaperonin containing TCP1, subunit 3 (gamma) (CCT3), transcript variant 1, |
| | | | mRNA |
| 410 | gi\|64085030 | 5395 | Homo sapiens PMS2 postmeiotic segregation increased 2 (S. cerevisiae) (PMS2), transcript |
| | | | variant 1, mRNA |
| 411 | gi\|66346686 | 27122 | Homo sapiens dickkopf homolog 3 (Xenopus laevis) (DKK3), transcript variant 1, mRNA |
| 412 | gi\|66472381 | 11188 | Homo sapiens nischarin (NISCH), mRNA |
| 413 | gi\|66932946 | 2 | Homo sapiens alpha-2-macroglobulin (A2M), mRNA |
| 414 | gi\|66933015 | 3615 | Homo sapiens IMP (inosine monophosphate) dehydrogenase 2 (IMPDH2), mRNA |
| 415 | gi\|67906819 | 7936 | Homo sapiens RD RNA binding protein (RDBP), mRNA |
| 416 | gi\|68160909 | 6208 | Homo sapiens ribosomal protein S14 (RPS14), transcript variant 3, mRNA |
| 417 | gi\|68216257 | 6143 | Homo sapiens ribosomal protein L19 (RPL19), mRNA |
| 418 | gi\|68533248 | 55859 | Homo sapiens brain expressed, X-linked 1 (BEX1), mRNA |
| 419 | gi\|6857798 | 10857 | Homo sapiens progesterone receptor membrane component 1 (PGRMC1), mRNA |
| 420 | gi\|68800342 | 3654 | Homo sapiens interleukin-1 receptor-associated kinase 1 (IRAK1), transcript variant 2, mRNA |
| 421 | gi\|6912481 | 3954 | Homo sapiens leucine zipper-EF-hand containing transmembrane protein 1 (LETM1), mRNA |
| 422 | gi\|69354670 | 23 | Homo sapiens ATP-binding cassette, sub-family F (GCN20), member 1 (ABCF1), transcript |
| | | | variant 1, mRNA |
| 423 | gi\|69885083 | 1877 | Homo sapiens E4F transcription factor 1 (E4F1), mRNA |
| 424 | gi\|70609879 | 3921 | Homo sapiens ribosomal protein SA (RPSA), transcript variant 1, mRNA |
| 425 | gi\|70778814 | 9581 | Homo sapiens prolyl endopeptidase-like (PREPL), mRNA |
| 426 | gi\|70906438 | 2266 | Homo sapiens fibrinogen gamma chain (FGG), transcript variant gamma-B, mRNA |
| 427 | gi\|71164870 | 10658 | Homo sapiens CUG triplet repeat, RNA binding protein 1 (CUGBP1), transcript variant 3, mRNA |
| 428 | gi\|71164880 | 6201 | Homo sapiens ribosomal protein S7 (RPS7), mRNA |
| 429 | gi\|71164882 | 7443 | Homo sapiens vaccinia related kinase 1 (VRK1), mRNA |
| 430 | gi\|71482588 | 6217 | Homo sapiens ribosomal protein S16 (RPS16), mRNA |
| 431 | gi\|71565153 | 128 | Homo sapiens alcohol dehydrogenase 5 (class III), chi polypeptide (ADH5), mRNA |
| 432 | gi\|71772514 | 6228 | Homo sapiens ribosomal protein S23 (RPS23), mRNA |
| 433 | gi\|72534753 | 112812 | Homo sapiens similar to RIKEN cDNA B230118G17 gene (MGC19604), transcript variant 1, |
| | | | mRNA |
| 434 | gi\|73695875 | 2483 | Homo sapiens FSHD region gene 1 (FRG1), mRNA |
| 435 | gi\|73747843 | 3980 | Homo sapiens ligase III, DNA, ATP-dependent (LIG3), nuclear gene encoding mitochondrial |
| | | | protein, transcript variant beta, mRNA |
| 436 | gi\|7382489 | 8694 | Homo sapiens diacylglycerol O-acyltransferase homolog 1 (mouse) (DGAT1), mRNA |
| 437 | gi\|74229006 | 3192 | Homo sapiens heterogeneous nuclear ribonucleoprotein U (scaffold attachment factor A) |
| | | | (HNRPU), transcript variant 2, mRNA |
| 438 | gi\|74229024 | 91304 | Homo sapiens chromosome 19 open reading frame 6 (C19orf6), transcript variant 1, mRNA |
| 439 | gi\|74271817 | 79415 | Homo sapiens chromosome 17 open reading frame 62 (C17orf62), mRNA |
| 440 | gi\|75813624 | 4773 | Homo sapiens nuclear factor of activated T-cells, cytoplasmic calcineurin-dependent 2 |
| | | | (NFATC2), transcript variant 1, mRNA |
| 441 | gi\|75992939 | 8237 | Homo sapiens ubiquitin specific peptidase 11 (USP11), mRNA |
| 442 | gi\|76150622 | 4839 | Homo sapiens nucleolar protein 1, 120 kDa (NOL1), transcript variant 1, mRNA |
| 443 | gi\|76257390 | 84196 | Homo sapiens ubiquitin specific peptidase 48 (USP48), transcript variant 1, mRNA |
| 444 | gi\|7657016 | 25832 | Homo sapiens neuroblastoma breakpoint family, member 14 (NBPF14), mRNA |
| 445 | gi\|7657121 | 50628 | Homo sapiens gem (nuclear organelle) associated protein 4 (GEMIN4), mRNA |
| 446 | gi\|7657133 | 8443 | Homo sapiens glyceronephosphate O-acyltransferase (GNPAT), mRNA |
| 447 | gi\|7657479 | 27069 | Homo sapiens growth hormone inducible transmembrane protein (GHITM), mRNA |
| 448 | gi\|7661727 | 28956 | Homo sapiens mitogen-activated protein-binding protein-interacting protein (MAPBPIP), mRNA |
| 449 | gi\|7661743 | 28969 | Homo sapiens basic leucine zipper and W2 domains 2 (BZW2), mRNA |
| 450 | gi\|7662309 | 9855 | Homo sapiens FERM, RhoGEF and pleckstrin domain protein 2 (FARP2), mRNA |
| 451 | gi\|7669500 | 3916 | Homo sapiens lysosomal-associated membrane protein 1 (LAMP1), mRNA |
| 452 | gi\|7706090 | 51283 | Homo sapiens bifunctional apoptosis regulator (BFAR), mRNA |
| 453 | gi\|7706588 | 2778 | Homo sapiens GNAS complex locus (GNAS), transcript variant 4, mRNA |
| 454 | gi\|77695910 | 7073 | Homo sapiens TIA1 cytotoxic granule-associated RNA binding protein-like 1 (TIAL1), transcript |
| | | | variant 1, mRNA |
| 455 | gi\|78000182 | 9045 | Homo sapiens ribosomal protein L14 (RPL14), transcript variant 1, mRNA |
| 456 | gi\|78190460 | 6147 | Homo sapiens ribosomal protein L23a (RPL23A), mRNA |
| 457 | gi\|78190461 | 6155 | Homo sapiens ribosomal protein L27 (RPL27), mRNA |
| 458 | gi\|78190465 | 6144 | Homo sapiens ribosomal protein L21 (RPL21), mRNA |
| 459 | gi\|78482615 | 2976 | Homo sapiens general transcription factor IIIC, polypeptide 2, beta 110 kDa (GTF3C2), transcript |
| | | | variant 1, mRNA |
| 460 | gi\|82546842 | 613 | Homo sapiens breakpoint duster region (BCR), transcript variant 1, mRNA |
| 461 | gi\|82617635 | 26999 | Homo sapiens cytoplasmic FMR1 interacting protein 2 (CYFIP2), transcript variant 2, mRNA |
| 462 | gi\|83376127 | 1933 | Homo sapiens eukaryotic translation elongation factor 1 beta 2 (EEF1B2), transcript variant 1, |
| | | | mRNA |
| 463 | gi\|83641893 | 3178 | Homo sapiens heterogeneous nuclear ribonucleoprotein A1 (HNRPA1), transcript variant 1, |
| | | | mRNA |
| 464 | gi\|83700221 | 9453 | Homo sapiens geranylgeranyl diphosphate synthase 1 (GGPS1), transcript variant 1, mRNA |
| 465 | gi\|83700234 | 1974 | Homo sapiens eukaryotic translation initiation factor 4A, isoform 2 (EIF4A2), mRNA |
| 466 | gi\|83779009 | 79048 | Homo sapiens SECIS binding protein 2 (SECISBP2), mRNA |
| 467 | gi\|86198309 | 123096 | Homo sapiens solute carrier family 25, member 29 (SLC25A29), transcript variant 1, mRNA |
| 468 | gi\|86198319 | 123096 | Homo sapiens solute carrier family 25, member 29 (SLC25A29), transcript variant 2, mRNA |
| 469 | gi\|86792636 | 533 | Homo sapiens ATPase, H+ transporting, lysosomal 21 kDa, V0 subunit b (ATP6V0B), transcript |
| | | | variant 1, mRNA |
| 470 | gi\|87159809 | 526 | Homo sapiens ATPase, H+ transporting, lysosomal 56/58 kDa, V1 subunit B2 (ATP6V1B2), |
| | | | mRNA |
| 471 | gi\|88758569 | 155400 | Homo sapiens NOL1/NOP2/Sun domain family, member 58 (NSUN5B), transcript variant 1, |
| | | | mRNA |
| 472 | gi\|88900496 | 23174 | Homo sapiens zinc finger, CCHC domain containing 14 (ZCCHC14), mRNA |
| 473 | gi\|88954355 | | Homo sapiens chromosome 2 genomic contig, reference assembly |
| 474 | gi\|88958353 | | Homo sapiens chromosome 2 genomic contig, alternate assembly (based on Celera assembly) |
| 475 | gi\|89034479 | | Homo sapiens chromosome 11 genomic contig, alternate assembly (based on Celera assembly) |
| 476 | gi\|89039160 | | Homo sapiens chromosome 15 genomic contig, alternate assembly (based on Celera assembly) |
| 477 | gi\|89040674 | | Homo sapiens chromosome 16 genomic contig, alternate assembly (based on Celera assembly) |
| 478 | gi\|89059047 | | Homo sapiens chromosome 22 genomic contig, reference assembly |
| 479 | gi\|89059140 | | Homo sapiens chromosome 22 genomic contig, alternate assembly (based on Celera assembly) |
| 480 | gi\|8922070 | 55565 | Homo sapiens hypothetical protein LOC55565 (LOC55565), mRNA |
| 481 | gi\|8922226 | 55068 | Homo sapiens proliferation-inducing protein 38 (PIG38), mRNA |
| 482 | gi\|8922727 | 55248 | Homo sapiens chromosome 1 open reading frame 75 (C1orf75), mRNA |
| 483 | gi\|8922734 | 55250 | Homo sapiens signal transducer and activator of transcription 3 interacting protein 1 (STATIP1), |
| | | | mRNA |
| 484 | gi\|8922866 | 55776 | Homo sapiens chromosome 6 open reading frame 64 (C6orf64), mRNA |
| 485 | gi\|8923416 | 54936 | Homo sapiens ADP-ribosylhydrolase like 2 (ADPRHL2), mRNA |
| 486 | gi\|89276753 | 2934 | Homo sapiens gelsolin (amyloidosis, Finnish type) (GSN), transcript variant 1, mRNA |
| 487 | gi\|89886487 | 9899 | Homo sapiens synaptic vesicle glycoprotein 2B (SV2B), mRNA |
| 488 | gi\|91208424 | 24148 | Homo sapiens PRP6 pre-mRNA processing factor 6 homolog (S. cerevisiae) (PRPF6), mRNA |
| 489 | gi\|91718898 | 5595 | Homo sapiens mitogen-activated protein kinase 3 (MAPK3), transcript variant 1, mRNA |
| 490 | gi\|91823780 | 389524 | Homo sapiens GTF2I repeat domain containing 2B (GTF2IRD2B), mRNA |
| 491 | gi\|92091570 | 83992 | Homo sapiens cortactin binding protein 2 (CTTNBP2), mRNA |
| 492 | gi\|92091577 | 11041 | Homo sapiens UDP-GlcNAc: betaGal beta-1,3-N-acetylglucosaminyltransferase 1 (B3GNT1), |
| | | | mRNA |
| 493 | gi\|92091602 | 116985 | Homo sapiens centaurin, delta 2 (CENTD2), transcript variant 2, mRNA |
| 494 | gi\|93141038 | 4899 | Homo sapiens nuclear respiratory factor 1 (NRF1), transcript variant 2, mRNA |
| 495 | gi\|93204870 | 59277 | Homo sapiens netrin 4 (NTN4), mRNA |
| 496 | gi\|93277120 | 83694 | Homo sapiens ribosomal protein S6 kinase-like 1 (RPS6KL1), mRNA |
| 497 | gi\|93277121 | 5936 | Homo sapiens RNA binding motif protein 4 (RBM4), mRNA |
| 498 | gi\|94538321 | 3029 | Homo sapiens hydroxyacylglutathione hydrolase (HAGH), transcript variant 1, mRNA |
| 499 | gi\|94538348 | 5217 | Homo sapiens profilin 2 (PFN2), transcript variant 1, mRNA |
| 500 | gi\|94721355 | 5447 | Homo sapiens P450 (cytochrome) oxidoreductase (POR), mRNA |
| 501 | gi\|9845292 | 9985 | Homo sapiens REC8-like 1 (yeast) (REC8L1), mRNA |
| 502 | gi\|9955964 | 11194 | Homo sapiens ATP-binding cassette, sub-family B (MDR/TAP), member 8 (ABCB8), nuclear |
| | | | gene encoding mitochondrial protein, mRNA |

1-18. (canceled)

19. A method for the diagnosis of pancreatic cancer diseases up to pancreatic carcinoma, comprising determining at least one marker sequence on or from a patient to be examined, wherein the at least one marker sequence comprises:
a) a cDNA selected from the group consisting of the nucleic acid sequences of SEQ ID NO: 1-1004;
b) a protein encoded by one of the nucleic acid sequences of SEQ ID NO: 1-1004; or
c) a partial sequence or fragment of the cDNA of a) or the protein of b).

20. The method of claim 19, wherein at least 2 marker sequences are determined on or from a patient to be examined.

21. The method of claim 19, wherein the determining at least one marker sequence on or from a patient to be examined is carried out by means of in-vitro diagnosis.

22. The method of claim 19, wherein the marker sequence is applied onto a solid support.

23. The method of claim 22, wherein the solid support is a filter, a membrane, a magnetic or fluorophore-labeled bead, a silica wafer, glass, metal, ceramics, plastics, a chip, a target for mass spectrometry, or a matrix.

24. A method for diagnosing pancreatic cancer diseases up to pancreatic carcinoma, comprising:
a) applying at least one marker sequence comprising:
i) a cDNA selected from the group consisting of the nucleic acid sequences of SEQ ID NO: 1-1004;
ii) a protein encoded by one of the nucleic acid sequences of SEQ ID NO: 1-1004; or
iii) a partial sequence or fragment of the cDNA of i) or the protein of ii).
b) contacting the at least one marker sequence of a) with body fluid or tissue extract of a patient; and
c) detecting an interaction of said body fluid or tissue extract with the marker sequence of a).

25. A method for the stratification, risk stratification, or therapy control of a patient with pancreatic cancer diseases up to pancreatic carcinoma, comprising determining on or from a patient to be examined at least one marker sequence comprising:
a) a cDNA selected from the group consisting of the nucleic acid sequences of SEQ ID NO: 1-1004;
b) a protein encoded by one of the nucleic acid sequences of SEQ ID NO: 1-1004; or
c) a partial sequence or fragment of the cDNA of a) or the protein of b).

26. The method of claim 25, wherein the stratification or the therapy control covers:
a) decisions for the treatment and therapy of the patient;
b) the hospitalization of the patient;
c) the use, effect and/or dosage of one or more drugs;
d) a therapeutic measure; or
e) the monitoring of a course of the disease and the course of therapy, etiology, or classification of a disease together with prognosis.

27. An arrangement of marker sequences containing at least one marker sequence comprising:
a) a cDNA selected from the group consisting of the nucleic acid sequences of SEQ ID NO: 1-1004;
b) a protein encoded by one of the nucleic acid sequences of SEQ ID NO: 1-1004; or
c) a partial sequence or fragment of the cDNA of a) or the protein of b).

28. The arrangement of claim 27, wherein the arrangement contains at least 2 marker sequences.

29. The arrangement of claim 27, wherein the marker sequences are present as clones.

30. An assay or protein biochip comprising the arrangement of claim 27, wherein the marker sequences are applied to a solid support.

31. A method for the identification and characterization of a substance for pancreatic cancer diseases up to pancreatic carcinoma containing means for detecting a binding success comprising:
a) contacting the arrangement of claim 27 with at least one substance to be tested: and
b) detecting a binding success.

32. A method for the identification and characterization of a substance for pancreatic cancer diseases up to pancreatic carcinoma comprising:
a) contacting the assay of claim 30 with at least one substance to be tested: and
b) detecting a binding success.

33. A method for screening active substances for pancreatic cancer diseases up to pancreatic carcinoma comprising utilizing the arrangement of claim 27.

34. A method for screening active substances for pancreatic cancer diseases up to pancreatic carcinoma comprising utilizing the assay of claim 30.

35. A diagnostic agent which comprises a sequence selected from the group consisting of:
a) the nucleic acid sequence of SEQ ID NO: 1-1004;
b) a protein sequence encoded by one of the nucleic acid sequences of SEQ ID NO: 1-1004, and
c) a partial sequence or fragment of the nucleic acid sequence of a) or the protein sequence of b).

36. A target for the treatment and therapy of pancreatic cancer diseases up to pancreatic carcinoma, wherein the target comprises a sequence selected from the group consisting of:
a) the nucleic acid sequence of SEQ ID NO: 1-1004;
b) a protein sequence encoded by one of the nucleic acid sequences of SEQ ID NO: 1-1004, and
c) a partial sequence or fragment of the nucleic acid sequence of a) or the protein sequence of b).

37. A method for carrying out an apheresis or blood lavage for patients with pancreatic cancer diseases up to pancreatic carcinoma, comprising contacting a body fluid of a patient with pancreatic cancer diseases up to pancreatic carcinoma with an affinity material comprising a marker sequence comprising:
a) a cDNA selected from the group consisting of the nucleic acid sequences of SEQ ID NO: 1-1004;
b) a protein encoded by one of the nucleic acid sequences of SEQ ID NO: 1-1004; or
c) a partial sequence or fragment of the cDNA of a) or the protein of b).
